(19) United States
(12) Patent Application Publication
(10) Pub. No.: US 2014/0303363 A1
(43) Pub. Date: Oct. 9, 2014

(54) Preparation and Use of Isolactosamine and Intermediates therefor

(71) Applicant: Glycom A/S, Kongens Lyngby (DK)

(72) Inventors: Markus Hederos, Svedala (SE); Christian Risinger, Rottweil (DE); Julien Boutet, La Plaine sur Mer (FR); Gyula Dekany, Sinnamon Park (AU)

(21) Appl. No.: 14/363,675

(22) PCT Filed: Dec. 7, 2012
(86) PCT No.: PCT/DK2012/050449

§ 371 Date: Jun. 6, 2014

(30) Foreign Application Priority Data
Dec. 9, 2011 (DK) PA 2011 70689

(51) Int. Cl. C07H 15/18; C07H 15/12; C07H 5/06

(52) U.S. Cl. CPC C07H 15/18; C07H 5/06; C07H 15/12
USPC 536/53; 536/18.7

(57) ABSTRACT

The invention relates to providing isolactosamine (Galβ1-3GlcNH₂, formula 1) and salts thereof in the form of either anomer or mixture thereof, as well as hydrates or solvates of the free base and salts thereof. The synthesis of isolactosamine and its use in the synthesis of lacto-N-biose containing oligosaccharides are also disclosed.

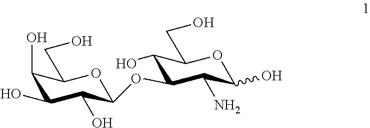

### FIELD OF THE INVENTION

The present invention relates to the provision and synthesis of a deoxyamino-disaccharide, namely isolactosamine.

### BACKGROUND OF THE INVENTION

Isolactosamine (Galβ1-3GlcNH₂, compound 1) is an important disaccharide which is a building block of glycoconjugates having wide range of biological properties.

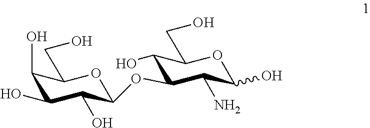

Isolactosamine is always N-acetylated in naturally occurring oligosaccharides and called lacto-N-biose (N-acetyl-isolactosamine, Galβ1-3GlcNAc). Lacto-N-biose is a structural part of the family of Le^{a}-type oligosaccharides. Sialylated and sulphated Le^{a} oligosaccharides were established as ligands for E-selectin, a carbohydrate binding protein which is expressed on cytokine-stimulated endothelial cells and plays important role in inflammation processes and in the haematogenous spread of cancer cells. Furthermore lacto-N-biose is a fundamental component of the terminal tetra- or pentasaccharides of Type 1 human ABH antigenic determinants. Lacto-N-biose containing oligosaccharides like lacto-N-tetraose and its fucosylated and/or sialylated derivatives are among some of the major components in human milk.

In the later years enormous progresses have been made in the production of lacto-N-biose containing oligosaccharides via enzymatic, biotechnological and chemical methodologies. However, most of these methodologies have not succeeded in large scale productions providing bulk quantities these oligosaccarides in a commercially attractive price range.

Enzymatic syntheses have developed significantly in the last decade by using enzyme cloning/mutating technologies. Wide range of enzymes enabled to use lacto-N-biose as donor or acceptor is required depending on the target oligosaccharide. Unfortunately, these enzymes are not commercially available in large quantities and therefore to date are not suitable for manufacturing technology developments. Some of them are rather sensitive and thus not really suitable for large scale preparations. Additionally, some of such enzymes require sugar nucleotide type donors which are hardly available and are extremely expensive.

Biotechnological methodologies use complex enzymatic systems facilitating both the biosynthesis of precursors and the required glycosylations. To date, such approaches face severe regulatory approval hurdles due to the use of genetically engineered organisms and potential contaminations of non-natural oligosaccharides.

Chemical syntheses have been until now still the most economically efficient way to obtain oligosaccharides having lacto-N-biose moiety. When conducting a multi-step reaction sequence some hurdles, however, especially in large scale chemical synthesis, may remain to be encountered such as low stereoselectivity, low overall yields, use of sophisticated and expensive purification methodologies like column chromatography, and the use of toxic reagents not suitable for food/therapeutic product developments. Thus providing improved methods and/or synthetically useful economic synthons is still a challenge in oligosaccharide synthesis.

The use of lacto-N-biose derivatives as glycosyl donors is rather limited due to the N-acetyl group present in the adjacent position to the anomeric centre. Once the anomeric position is activated for glycosylation, a fused oxazoline is formed by the attack of the acetyl's carbonyl to the glycosidic centre. The fused oxazoline is still a glycosylating agent but—owing to its diminished reactivity—the glycosylation reaction runs with lower to poor yields. In order to improve the yield stronger promoter(s) may be applied, however the protecting groups of the reactants may be affected. Application of more forcing reaction conditions (higher temperature, longer reaction time, etc.) may favour by-product formation and spoil regio- and/or stereoselectivity. Using large excess of acceptor may entail separation and isolation difficulties.

In order to eliminate the above-mentioned disadvantages to prepare isolactosamine derivatives suitable as glycosyl donor for oligosaccharide synthesis, numerous attempts with different protecting groups were made. Generally, the interglycosidic linkages are formed by glycosylation reactions, most commonly a promoter-assisted nucleophilic displacement of the leaving group of the glycosyl donor with the hydroxyl moiety of the glycosyl acceptor. Other functional groups on both the donor and the acceptor are temporarily masked with protecting groups. As the isolactosamine (or lacto-N-biose) portion can be either at the terminal positions of or within a (often highly branched) polysaccharide chain, large number of such protected key intermediates are described ready for coupling which bear widely manifold protecting groups depending on whether they will be applied to as donor, acceptor or both.

The main drawback of the above-mentioned procedures is the unavoidable chromatographic separation in order either to get the pure substance or to obtain at least a mixture that is enriched in the target compound but still contains undesired derivatives. Although repeated chromatographic separation may results in the improvement of the purity, its high cost and relatively long technological time to handle the feed solution and the column packing, to carry out the separation and optionally to regenerate the packing, especially in large or industrial scale, can be disadvantageous and/or cumbersome.

Crystallization or recrystallization is one of the simplest and cheapest methods to isolate a product from a reaction mixture, separate it from contaminations and obtain pure substance. Isolation or purification that uses crystallization makes the whole technological process robust and cost-effective, thus it is advantageous and attractive compared to other procedures.

Isolactosamine and salts thereof have not been described yet by the prior art. Its 1-O-α-benzyl derivative was selected as model bioside for developing a standard procedure to cleave the interglycosidic linkage selectively (Dmitriev et al. Carb. Res. 29, 451 (1973)). The β-trichloroethyl glycoside and its hydrochloride salt were used as standard for conformation analysis of Lewis human blood group determinant oligosaccharides in NMR investigations (Lemieux et al. Can. J. Chem. 58, 631 (1980)). The β-(8-methoxycarbonyloctyl) glycoside is also characterized (Baisch et al. Carb. Res. 312, 61 (1998)).

The present inventors provide unprotected isolactosamine and salts thereof as disaccharide building block for the synthesis of oligosaccharides having lacto-N-biose structural part. The salts are available in crystalline form, can be handled easily and offer a general and highly pure starting material for preparing suitable armed isolactosamine synthons towards oligosaccharide production.

### SUMMARY OF THE INVENTION

In accordance with the present invention, there is provided isolactosamine (Galβ1-3GlcNH₂) of formula 1 and salts thereof in the form of either anomer or mixture thereof, as well as hydrates or solvates of the free base and salts thereof.

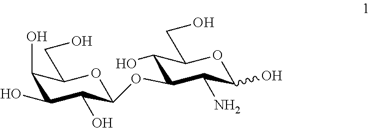

According to the second aspect the present invention provides a method for the preparation of isolactosamine of formula 1 and salts thereof in the form of either anomer or mixture thereof, as well as hydrates or solvates of the free base and salts thereof. The method is based upon the utilisation of isolactosamine derivatives of general formula 2 or salts thereof in the form of either anomer or mixture thereof, as well as hydrates or solvates of the free base and salts thereof, wherein R is selected from H and a group that can be removed by hydrogenolysis, and R₁ is selected from azido and —NHR₂ wherein R₂ is selected from H, optionally substituted benzyloxycarbonyl and optionally substituted benzyloxymethyl, provided if R is H then R₁ differs from —NH_{2,} in a catalytic hydrogenation/hydrogenolysis reaction.

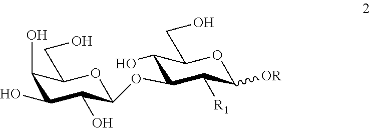

The third aspect of the present invention is to provide compounds of general formula 2 and salts thereof in the form of either anomer or mixture thereof, as well as hydrates or solvates of the free base and salts thereof wherein R is selected from H and a group that can be removed by hydrogenolysis, and R₁ is selected from azido and —NHR₂ wherein R₂ is selected from H, optionally substituted benzyloxycarbonyl and optionally substituted benzyloxymethyl, provided if R is H then R₁ differs from —NH₂, and further provided that benzyl 3-O-(β-D-galactopyranosyl)-2-deoxy-2-amino-α-D-glucopyranoside is excluded.

The fourth aspect of the invention relates to the use of isolactosamine and salts thereof according to the first aspect for the preparation of lacto-N-biose containing oligosaccharides. The fifth aspect relates to a method for synthesis of lacto-N-biose containing oligosaccharides, comprising the steps of:
a) masking of the amino group of isolactosamine with a suitable protecting group,
b) protection of OH-groups,
c) activation the anomeric position to obtain a lacto-N-biosyl donor, and
d) coupling the lacto-N-biosyl donor to a desired sugar moiety.

### BRIEF DESCRIPTION OF THE FIGURE

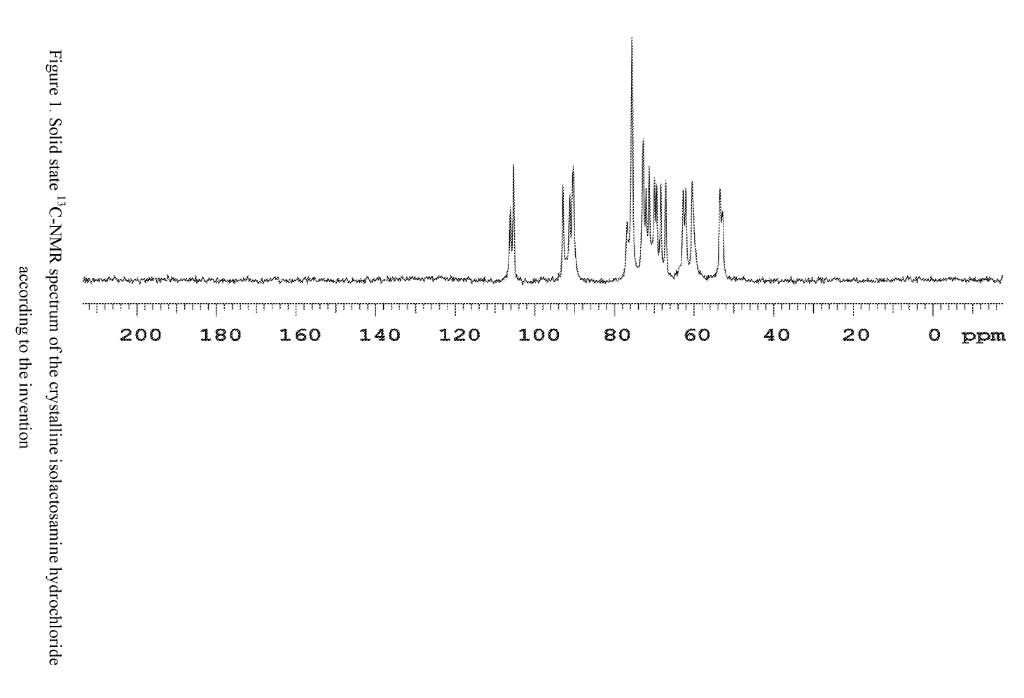

The invention will be described in further detail hereinafter with reference to the accompanying FIG. 1, which shows the solid state ¹³C-NMR spectrum of the crystalline isolactosamine hydrochloride according to the present invention.

### DETAILED DESCRIPTION OF THE INVENTION

Throughout the present description, the term “alkyl” means a linear or branched chain saturated hydrocarbon group with 1-6 carbon atoms, such as methyl, ethyl, n-propyl, i-propyl, n-butyl, i-butyl, s-butyl, t-butyl, n-hexyl, etc.

The term “aryl” refers to a homoaromatic group such as phenyl or naphthyl.

In the present description, the term “acyl” represents an Q-C(═O)-group, wherein Q may be H, alkyl (see above) or aryl (see above), such as formyl, acetyl, propionyl, butyryl, pivaloyl, benzoyl, etc.

“Benzyl” is phenylmethyl.

The term “alkyloxy” or “alkoxy” means an alkyl group (see above) attached to the parent molecular moiety through an oxygen atom, such as methoxy, ethoxy, t-butoxy, etc.

“Halogen” means fluoro, chloro, bromo or iodo.

“Haloalkyl” means alkyl substituted by at least one halogen, such as chloromethyl, trichloromethyl, trifluoromethyl, 2,2,2-trichloroethyl, etc.

“Amino” refers to a —NH₂ group.

“Alkylamino” means an alkyl group (see above) attached to the parent molecular moiety through an —NH-group, such as methylamino, ethylamino, etc.

“Dialkylamino” means two alkyl groups (see above), either identical or different ones, attached to the parent molecular moiety through a nitrogen atom, such as dimethylamino, diethylamino, etc.

“Acylamino” or “acylamido” or “amido” refer to an acyl group (see above) attached to the parent molecular moiety through an —NH-group, such as acetylamino (acetamido), benzoylamino (benzamido), etc.

“Alkanoylamido” or “alkanoylamino” means an acylamino group having alkyl chain, such as formamido, acetylamino (acetamido), propionyalmino (propionamido), etc.

“Haloalkanoylamido” or “haloalkanoylamino” refers to acylamino having haloalkyl chain, like trichloroacetamido, trifluoroacetamido, etc.

“Carboxyl” denotes an —COOH group.

“Alkyloxycarbonyl” means an alkyloxy group (see above) attached to the parent molecular moiety through a —C(═O)-group, such as methoxycarbonyl, t-butoxycarbonyl, etc.

“Haloalkyloxycarbonyl” means an haloalkyloxy group attached to the parent molecular moiety through a —C(═O)-group, such as 2,2,2-trichloroethoxycarbonyl and the like.

“Carbamoyl” is an H₂N—C(═O)-group.

“N-Alkylcarbamoyl” means an alkyl group (see above) attached to the parent molecular moiety through a —HN—C(═O)-group, such as N-methylcarbamoyl, etc.

“N,N-Dialkylcarbamoyl” means two alkyl groups (see above), either identical or different ones, attached to the parent molecular moiety through a >N—C(═O)-group, such as N,N-methylcarbamoyl, etc.

The alkyl or aryl residues in any of the above-mentioned groups may either be unsubstituted or may be substituted with one or more groups selected from alkyl (only for aryl residues), halogen, nitro, aryl, alkoxy, amino, alkylamino, dialkylamino, acylamino, carboxyl, alkoxycarbonyl, carbamoyl, N-alkylcarbamoyl, N,N-dialkylcarbamoyl, azido, halogenalkyl or hydroxyalkyl, giving rise to acyl groups such as chloroacetyl, trichloroacetyl, 4-chlorobenzoyl, 4-nitrobenzoyl, 4-phenylbenzoyl, etc., and referred to as “optionally substituted”.

In accordance with the present invention, there is provided isolactosamine (Galβ1-3GlcNH₂) of formula 1 and salts thereof in the form of either anomer or mixture thereof, as well as hydrates or solvates of the free base and salts thereof.

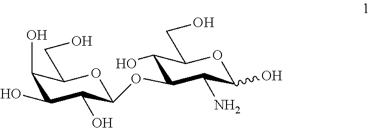

Novel isolactosamine of formula 1 and salts thereof can be characterized as crystalline solids, oils, syrups, precipitated amorphous material or spray dried products. If crystalline, they might exist either in anhydrous or in hydrated crystalline forms by incorporating one or several molecules of water into their crystal structures. Similarly, isolactosamine and salts thereof might exist as crystalline substances incorporating ligands such as organic molecules and/or ions into their crystal structures. The crystalline form of isolactosamine and salts thereof can be considered as an anomeric mixture of α- and β-anomers or even pure form of one of the anomers.

Preferably, novel isolactosamine of formula 1 is characterized as salt. Suitable inorganic acids forming salt with isolactosamine include, but are not limited to, HCl, H₂SO₄, HNO₃ and H₃PO₄. Suitable organic acids forming salt with isolactosamine include, but are not limited to, formic acid, acetic acid and oxalic acid. The salts of the aforementioned acids with a base whose basicity is weaker than that of isolactosamine can be suitable as well for forming salt with isolactosamine. More preferably the salts of isolactosamine of formula 1 are crystalline. Even more preferably, the salt of isolactosamine of formula 1 is the hydrochloride salt.

According to the second aspect the present invention provides a method for the preparation of isolactosamine of formula 1 and salts thereof in the form of either anomer or mixture thereof, as well as hydrates or solvates of the free base and salts thereof. The method is based upon the utilisation of isolactosamine derivatives of general formula 2 or salts thereof in the form of either anomer or mixture thereof, as well as hydrates or solvates of the free base and salts thereof, wherein R is selected from H and a group that can be removed by hydrogenolysis, and R₁ is selected from azido and —NHR₂ wherein R₂ is selected from H, optionally substituted benzyloxycarbonyl and optionally substituted benzyloxymethyl, provided if R is H then R₁ differs from —NH₂, in a catalytic hydrogenation/hydrogenolysis reaction.

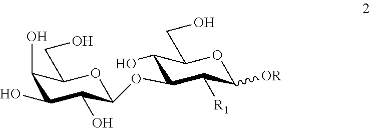

In detail, the protecting group that is removable by hydrogenolysis or can be removed by hydrogenolysis refers to groups whose bond attached to the grouping to be masked is cleaved by addition of hydrogen in the presence of catalytic amounts of palladium, Raney nickel or another appropriate metal catalyst known for use in hydrogenolysis, resulting in the regeneration of the group. Such protecting groups are well known to the skilled person and are discussed e.g. in Protective Groups in Organic Synthesis, P G M Wuts and T W Greene, John Wiley & Sons 2007. Suitable protecting groups include (without limitation) benzyl, diphenylmethyl(benzhydryl), 1-naphthylmethyl, 2-naphthylmethyl, benzyloxymethyl, benzyloxycarbonyl or triphenylmethyl(trityl) groups, each of which may be optionally substituted by one or more groups selected from: alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl or halogen. Preferably, such substitution, if present, is on the aromatic ring(s). Particularly preferred protecting groups are benzyl or naphthylmethyl groups optionally substituted with one or more groups selected from phenyl, alkyl or halogen. More preferably, the protecting group is selected from unsubstituted benzyl, unsubstituted 2-naphthylmethyl, 4-chlorobenzyl, 3-phenylbenzyl and 4-methylbenzyl. These particularly preferred and more preferable protecting groups have the advantage that the by-products of the hydrogenolysis are exclusively toluene, 2-methylnaphthalene, or substituted toluene or 2-methylnaphthalene derivatives, respectively. Such by-products can easily be removed even in multi ton scales from the water soluble isolactosamine via evaporation and/or extraction processes.

The term catalytic hydrogenolysis or catalytic hydrogenation intends to mean reduction with hydrogen in the presence of a catalyst (see above) that typically takes place in a protic solvent or in a mixture of protic solvents. A protic solvent may be selected from a group consisting of water, acetic acid or C₁-C₆ alcohol. Mixture of one or more protic solvents with one or more proper aprotic organic solvents miscible partially or fully with the protic solvent(s) (such as THF, dioxane, ethyl acetate, acetone, etc.) may also be applied. Water, one or more C₁-C₆ alcohols or a mixture of water and one or more C₁-C₆ alcohols are preferably used as solvent system. Solutions containing the carbohydrate derivatives in any concentration or suspensions of the carbohydrate derivatives with the solvent(s) used are also applicable. The reaction mixture is stirred at 10-100° C. temperature range, preferably between 20-50° C. in hydrogen atmosphere of 1-50 bar in the presence of a catalyst such as palladium, Raney nickel or any other appropriate metal catalyst, preferably palladium on charcoal or palladium black, until reaching the completion of the reaction. Catalyst metal concentrations generally range from 0.1% to 10% based on the weight of carbohydrate. Preferably, the catalyst concentrations range from 0.15% to 5%, more preferably 0.25% to 2.25%. Transfer hydrogenolysis may also be performed, when the hydrogen is generated in situ from cyclohexene, cyclohexadiene, formic acid or ammonium formate. Addition of organic or inorganic bases/acids and/or basic and/or acidic ion exchange resins can also be used to improve the kinetics of the hydrogenolysis. The use of basic substances is especially preferred when halogen substituents are present on the substituted benzyl moieties of the precursors and/or the formation of isolactosamine base is desirable. Preferred organic bases are including but not limited to triethylamine, diisopropyl ethylamine, ammonia, ammonium carbamate, diethylamine, etc. Acid is favourably used as a co-solvent or additive in cases when multiple benzyl groups have to be removed from the precursor and/or isolactosamine salts is intended to isolate. Preferred acids are including but not limited to formic acid, acetic acid, propionic acid, chloroacetic acid, dichloroacetic acid, trifluoroacetic acid, HCl, HBr, etc. The conditions proposed allow simple, convenient and delicate removal of the solvent(s) giving rise to isolactosamine.

In a preferred embodiment a compound of general formula 2, wherein R is benzyl, 2-naphthylmethyl, 4-chlorobenzyl, 3-phenylbenzyl and 4-methylbenzyl, more preferably benzyl, and R₂ is —NH₂, or a salt thereof is subjected to catalytic hydrogenolysis to provide isolactosamine or a salt thereof. The catalytic hydrogenolysis can be performed in water or in aqueous alcohol, preferably in water, water/methanol or water/ethanol mixture (alcohol content: 10-50 v/v %). The catalytic hydrogenolysis is performed at a temperature of between 15-65° C., preferably between 25-40° C.

If isolactosamine base is formed in the reductive step it may be converted into its acid-addition salt. The salt formation is typically carried out in solution using inorganic or organic acids or salts. Solvents including but not limited to acetone, methanol, ethanol, water, dioxane, DMSO, THF, DMF, alcohols, MeCN, and the mixtures of thereof can be used for such transformation. Inorganic acids are including but not limited to HCl, H₂SO₄, HNO₃, H₃PO₄, etc. in concentrated or diluted in water or any other solvents such as methanol, ethanol, dioxane, etc. Organic acids are including but not limited to formic acid, acetic acid, oxalic acid, etc. The salts of these acids with a base whose basicity is weaker than that of isolactosamine can be used as well. Products are typically obtained by selective precipitation adding apolar solvents like diethyl ether, diisopropyl ether, acetone, ethanol, isopropanol etc. or by crystallization in high yield without any chromatography.

The third aspect of the present invention is to provide compounds of general formula 2 and salts thereof in the form of either anomer or mixture thereof, as well as hydrates or solvates of the free base and salts thereof wherein R is selected from H and a group that can be removed by hydrogenolysis, and R₁ is selected from azido and —NHR₂ wherein R₂ is selected from H, optionally substituted benzyloxycarbonyl and optionally substituted benzyloxymethyl, provided if R is H then R₁ differs from —NH₂, and further provided that benzyl 3-O-(β-D-galactopyranosyl)-2-deoxy-2-amino-α-D-glucopyranoside is excluded. Compounds of general formula 2 and salts thereof can be characterized as crystalline solids, oils, syrups, precipitated amorphous material or spray dried products. If crystalline, they may exist either in anhydrous or in hydrated crystalline forms by incorporating one or several molecules of water into their crystal structures. Similarly, compounds of general formula 2 and salts thereof may exist as crystalline substances incorporating ligands such as organic molecules and/or ions into their crystal structures. Compounds of general formula 2 and salts thereof include anomeric mixtures of α- and β-anomers and/or the pure form of the α- or the β-anomers.

Preferred compounds falling into the scope of general formula 2 are characterized by general formula 2′

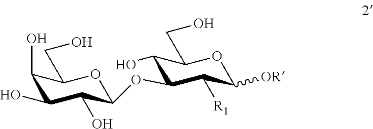

wherein R′ is a group that can be removed by hydrogenolysis, and R₁ is selected from azido and —NHR₂ wherein R₂ is selected from H, optionally substituted benzyloxycarbonyl and optionally substituted benzyloxymethyl. In more preferred compounds R′ is naphthylmethyl or benzyl optionally substituted by alkyl, halogen or phenyl, and R₁ is amino or benzyloxycarbonylamino. In particularly preferred compounds of general formula 2′ R′ is benzyl, 4-chlorobenzyl, 3-phenylbenzyl or 4-methylbenzyl, and R₁ is amino.

The chemistry necessary to prepare compounds of general formula 2′ is well known in the field of carbohydrate chemistry. A possible pathway is outlined in Scheme 1.

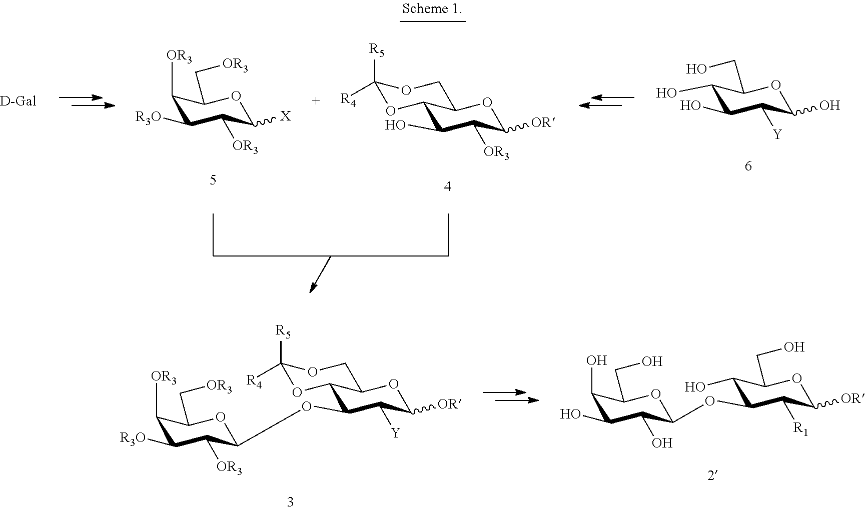

Donors of general formula 5 wherein R₃ means optionally substituted acyl can be easily prepared by known methods. Glycosyl iodides, bromides and chlorides (X═I, Br, Cl) can be synthesized by treatment of the readily available peracylated galactose with appropriate halogenating agent (e.g. hexamethyl-disilazane/I₂, trimethyl iodosilane, Et₃SiH/I₂, HBr, PBr₃, thionyl chloride, PCl₅/BF₃-etherate, TiCl₄, etc.). The glycosyl fluorides (X═F) may be prepared by treatment of the appropriate precursors such as hemiacetals, glycosyl halides, glycosyl esters and S-glycosides with fluorinating reagents such as HF, AgF, AgBF₄, tetrabutyl ammonium fluoride, diethylaminosulfur trifluoride, 2-fluoro-1-methylpyridinium tosylate, Selectfluor, Deoxo-Fluor, 4-methyl(difluoroiodo)benzene, etc. Trichloroacetimidates (X═—OC(═NH)CCl₃) can be easily obtained by the addition of the free anomeric OH of the protected hemiacetal to trichloroacetonitrile under inorganic or organic base catalysis. The pentenyl glycosides (X means —O—(CH₂)₃—CH═CH₂) can be prepared with the aid of n-pentenol by standard Fischer glycosylation of hemiacetals under acidic condition, by silver(I) salt promoted coupling of glycosyl bromides (Koenigs-Knorr method), or by glycosylation of 1-acetyl glycosides in the presence of tin(IV) chloride. Thioglycosides (X═—SR₆, in which R₆ is alkyl or optionally substituted phenyl) can be achieved by thiolysis of peracylated galactose with R₆SH in the presence of a Lewis acid, or by reacting halo compounds (X═Cl, Br) in phase transfer catalysed reactions with a base, R₆SH and a quaternary ammonium salt.

The synthesis of acceptors of general formula 4 (wherein R′ is a group removable by hydrogenolysis, R₄ represents alkyl or optionally substituted phenyl, R₅ means H, alkyl or optionally substituted phenyl and Y is selected from alkanoylamido, haloalkanoylamido, alkoxycarbonylamino, haloalkoxycarbonylamino, optionally substituted benzyloxycarbonylamino, optionally substituted benzyloxymethylamino, —NAc₂, optionally substituted benzamido, phthalimido, tetrachlorophthalimido, 2,3-diphenylmaleimido, 2,3-dimethylmaleimido and azido) may be performed as follows. D-Glucosamine is N-acylated lege artis or reacted with alkoxycarbonyl, haloalkoxycarbonyl, benzyloxycarbonyl or benzyloxymethyl halogenide, preferably chloride under base catalysis to compounds of general formula 6 wherein Y is selected from alkanoylamido, haloalkanoylamido, alkoxycarbonylamino, haloalkoxycarbonylamino, optionally substituted benzyloxycarbonylamino, optionally substituted benzyloxymethylamino, —NAc₂, optionally substituted benzamido, phthalimido, tetrachlorophthalimido, 2,3-diphenylmaleimido and 2,3-dimethylmaleimido. 2-Azido-2-deoxy-D-glucose (Y is azido) is readily available. The reaction of the D-glucosamine derivatives of general formula 6 and R′OH under Fischer glycosidation condition or in the presence of trimethyl chlorosilane readily results in preferably the α-glucoside, whereas treatment with R′-halogenide in the presence of a hydride yields preferably the β-glucoside derivative. Alternatively, acetonidation of N-acetyl-D-glucosamine (acetone/BF₃-etherate, reflux) gives a 5,6-O-isopropylidene glucofuranose oxazoline derivative, which is then allowed to react with R′OH in the presence of a strong acid providing the β-2-deoxy-2-acetamido-D-glucopyranoside. Then, the cyclic acetal/ketal formation can be conducted with an aldehyde or ketone of general formula R₄R₅(C═O) or dimethyl acetals thereof (R₄ represents alkyl or optionally substituted phenyl and R₅ means H, alkyl or optionally substituted phenyl) in the presence of protic (e.g. HCl, HBr, sulphuric acid, sulphonic acids) or Lewis acids (e.g. AlCl₃, FeCl₃, SnCl₂, ZnCl₂) giving rise to compounds of general formula 4.

A glycosyl donor of general formula 5 (wherein X is halogen, —OC(═NH)CCl₃, —O-pentenyl, —OAc, —OBz or —SR₆, in which R₆ is alkyl or optionally substituted phenyl, and R₃ means optionally substituted acyl) are reacted with a glycosyl acceptor of general formula 4 (wherein R′ is a group removable by hydrogenolysis, R₄ represents alkyl or optionally substituted phenyl, R₅ means H, alkyl or optionally substituted phenyl and Y is selected from alkanoylamido, haloalkanoylamido, alkoxycarbonylamino, haloalkoxycarbonylamino, optionally substituted benzyloxycarbonylamino, optionally substituted benzyloxymethylamino, —NAc₂, optionally substituted benzamido, phthalimido, tetrachlorophthalimido, 2,3-diphenylmaleimido, 2,3-dimethylmaleimido and azido) under glycosylation condition. The term “glycosidation condition” means in the present context to run the reaction in an aprotic solvent or in a mixture of aprotic solvents in the presence of an activator so as to lead to the desired glycosylated product by controlling the stereoselectivity of the conjugation via non-neighbouring group active protecting group strategy, solvent effect, halide effect, promoter selection and temperature control. In case of carbohydrates an array of anomeric activation for glycosylation is developed and available to a skilled person engaged in synthetic carbohydrate chemistry. These methodologies are expansively discussed by reviews and handbooks, for instance by Demchenko (Ed.): Handbook of Chemical Glycosylation Wiley (2008). For the sake of examples some general considerations are briefly mentioned below depending on the X-group (the protecting groups of the acceptors and donors remain intact under glycosylation).

The glycosyl halides (X means F, Cl, Br, I) are frequently used in glycosylation reaction because of their easy accessibility and satisfactory reactivity. Typically, anomeric halides follow the reactivity order F<Cl<Br<I for nucleophilic displacement. The glycosylation reactions are generally promoted by heavy metal ion, mainly mercury or silver, and Lewis acids. Glycosyl trichloroacetimidates (X═—OC(═NH)CCl₃) in a typical glycosidation reactions can be promoted by catalytic amount of Lewis acid, such as trimethylsilyl triflate or BF₃-etherate. Glycosyl acetates or benzoates (X represents —OAc or —OBz) in glycosylation reactions are first subjected to electrophilic activation providing a reactive intermediate, then treated with the nucleophilic OH-acceptor. Typical activators of choice are Bronsted acids (such as p-TsOH, HClO₄, sulfamic acid, etc.), Lewis acids (such as ZnCl₂, SnCl₄, triflate salts, BF₃-etherate, trityl perchlorate, AlCl₃, triflic anhydride, etc.) and their mixtures. Pentenyl glycosides (X means —O—(CH₂)₃—CH═CH₂) as glycosyl donors can be transglycosylated with appropriate glycosyl acceptors in the presence of a promoter such as NBS and NIS. Protic or Lewis acids (triflic acid, Ag-triflate, etc.) may enhance the reaction. Thioglycosides (X denotes alkylthio- or optionally substituted phenylthio-group) can be activated by thiofilic promoters such as mercury(II) salts, Br₂, I₂, NBS, NIS, triflic acid, triflate salts, BF₃-etherate, trimethylsilyl triflate, dimethyl-methylthio sulphonium triflate, phenylselenyl triflate, iodonium dicollidine perchlorate, tetrabutylammonium iodide or mixtures thereof, in condensation reactions, preferably by Br₂, NBS, NIS or triflic acid.

The different types of protecting groups on compounds of general formula 3 may be removed successively. Thus, R₃-group as optionally substituted acyl can be split by base catalysed transesterification reaction that is the acyl protective groups from hydroxyls are removed in an alcohol solvent such as methanol, ethanol, propanol, t-butanol, etc. in the presence of an alcoholate like NaOMe, NaOEt, KO^{t}Bu, etc. at 20-100° C. temperatures. The alcohol and the alcoholate should be matched. The use of co-solvent as toluene or xylene might be beneficial in order to avoid gel formations. In a preferred embodiment catalytic amount of NaOMe is used in methanol (Zemplén deacylation). If Y is —NAc₂, under the condition of base catalysed transesterification one of the acyl groups is also removed to give a compound having a —NHAc substituent as Y.

The cyclic acetal/ketal may be deprotected by acid catalysed mild hydrolysis, that is by the reaction in which water or alcohol reacts in the presence of acid at pH>1-2 with a substance bearing acid labile protective group(s) to regenerate the OH-group(s) protected. The starting compound may contain acyl protective groups as well. The skilled person is fully aware that acyl groups can be deprotected by only extremely strong acidic hydrolysis (pH<1). The skilled person is able to distinguish which deprotective condition affects the acetal/ketal group while the acyl groups remain intact. Furthermore the interglycosidic linkage and the aglycon of the glucosamine portion may be also sensitive to acids. The skilled person is fully aware that interglycosidic linkages and anomeric protecting groups can be split by only strong acidic hydrolysis (pH<1-2). The skilled person is able to distinguish which deprotective condition affects the acetal/ketal group while the interglycosidic linkages remain intact. Water—which has to be present in the reaction milieu as reagent—may serve as solvent or co-solvent as well. Organic protic or aprotic solvents which are stable under acidic conditions and miscible fully or partially with water such as C₁-C₆ alcohols, acetone, THF, dioxane, ethyl acetate, MeCN, etc. may be used in a mixture with water. The acids used are generally protic acids selected from but not limited to acetic acid, trifluoroacetic acid, HCl, formic acid, sulphuric acid, perchloric acid, oxalic acid, p-toluenesulfonic acid, benzenesulfonic acid, cation exchange resins, etc., which may be present in from catalytic amount to large excess. The hydrolysis may be conducted at temperatures between 20° C. and reflux until reaching completion which takes from about 2 hours to 3 days depending on temperature, concentration and pH. Preferably, organic acids including but not limited to aqueous solutions of acetic acid, formic acid, chloroacetic acid, oxalic acid, etc. are used. Another preferred condition is to use a C₁-C₆ alcohol-acetonitrile or C₁-C₆ alcohol-water mixture in the presence of HCl or sulfonic acids such as p-toluenesulfonic acid or camphorsulfonic acid. Alternatively, anhydrous C₁-C₆ alcohol including but not limited to methanol, ethanol, propanol, butanol, etc. can also be used for the required cleavage of the cyclic acetal/ketal moieties via acid catalysed trans-acetalization/trans-ketalization processes. Catalytic amount of hydrogen chloride, sulphuric acid, perchloric acid, p-toluenesulfonic acid, acetic acid, oxalic acid, camphorsulfonic acid, strong acidic ion-exchange resins, etc. can be used for the purposes at temperatures of 20° C. to reflux. Under the condition described above t-butoxycarbonylamino as Y-group may be deprotected to amino.

The acyl group on the amino portion (Y is optionally substituted acylamido) may be removed by basic hydrolysis which generally means base catalysed hydrolysis in water, alcohol or water-organic solvent mixtures, in homogeneous or heterogeneous reaction conditions at temperatures varying from 0-100° C., preferably in reflux temperature. The base of choice is generally a strong base, e.g. LiOH, NaOH, KOH, Ba(OH)₂, K₂CO₃, basic ion exchange resins, tetraalkylammonium hydroxides, etc. The bases can be used in the form of an aqueous solution as well. This condition affects O-acyls, alkoxycarbonylamino, 2,3-diphenylmaleimide and 2,3-dimethylmaleimide as well. An alternative way to deacylate may be aminolysis (N-acyl transfer based deprotection) which means a treatment with ammonia, hydrazine, substituted hydrazine, ethylene diamine or primary amines in water, alcohol or water-organic solvent mixtures at 20-120° C. temperatures. Under this condition all of the O- and N-protecting acyl groups, including cyclic imides and carbamate type groups can be readily removed.

2,2,2-Trichloroethoxycarbonylamino as Y-group can be converted into amino by means of Zn/HCl.

All combination of methods or process steps as used herein to deprotect compounds of general formula 3 can be performed in any order giving rise to compounds of general formula 2′.

Another preferred compounds falling into the scope of general formula 2 are characterized by general formula 2″

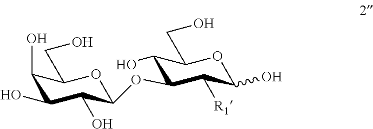

wherein R₁′ is selected from azido and —NHR₂′ wherein R₂′ is selected from optionally substituted benzyloxycarbonyl and optionally substituted benzyloxymethyl.

Compounds characterized by general formula 2″ can be synthesized in the way described below (see Scheme 2).

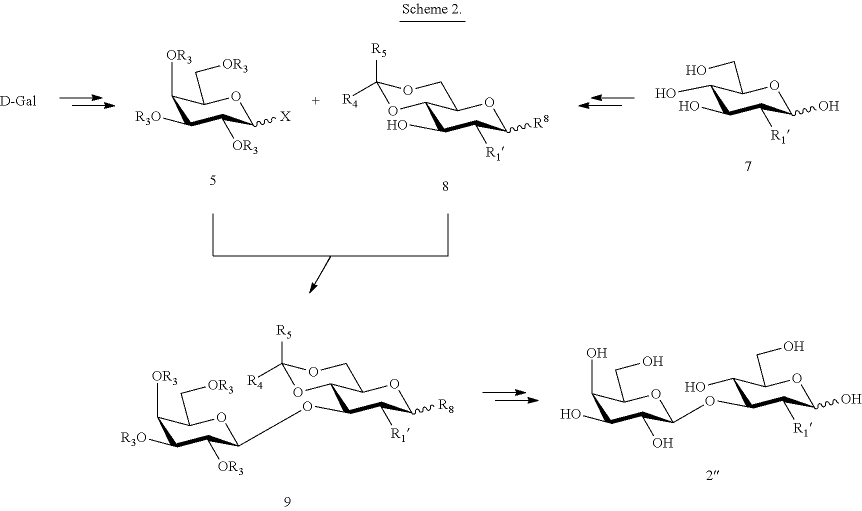

Compounds of general formula 7 wherein R₁′ is defined above, are readily available or can be made as described above. Peracetylation with acetic anhydride in the presence of base followed by Lewis acid mediated thiolysis with a thiol of formula R₇SH (wherein R₇ is alkyl or optionally substituted phenyl) gives rise to the thioglycoside derivative, which are converted into compounds of general formula 8, wherein R₄ represents alkyl or optionally substituted phenyl, R₅ means H, alkyl or optionally substituted phenyl, and R₈ is —SR₇, upon Zemplén-deacetylation and acetalation/ketalation as described above. Compounds of general formula 8 in which R₈ means acyloxy group can be prepared by the following two set sequence: compounds of general formula 7 are brought to acetal/ketal formation as described above then the free anomeric OH-group is acylated by 1-acyloxy-1H-benztriazole or 1-acyloxy-1H-4,5,6,7-tetrachlorobenztriazole. Glycosylation of acceptor of general formula 8 with donor of general formula 5 under the conditions specified above affords compounds of general formula 9, wherein R₁′, R₃, R₄, R₅ and R₈ are as defined above. The anomeric OH can be set free by base catalysed transesterification reaction or basic hydrolysis (when R₈ is acyloxy), or by treatment with a thiophilic activator such as mercury(II) salts, Br₂, I₂, NBS, NIS, triflic acid or triflate salts, or mixtures thereof in the presence of water (when R₈ is —SR₇). Removal of the other protective groups of compounds of general formula 9 can be carried out as specified above. The skilled person is able to select the proper deprotective conditions and to determine the order of the deprotective step which leads to compounds of general formula 2″.

According to a further aspect of the invention, isolactosamine and salts thereof may serve as useful synthons for the synthesis of oligosaccharides containing Gaβ1-3GlcNAc moiety. One of most crucial points is the availability of isolactosamine donor having a masking group on the amino group. The suitable protective groups may be, for instance, phthalyl, tetrachlorophthalyl, dithiasuccinoyl, trichloroacetyl, trifluoroacetyl, dimethylmaleolyl, benzyloxycarbonyl, trichloroethoxycarbonyl or allyloxycarbonyl group, all of them are readily used to protect the amino function in glycosylation reactions (see e.g. Aly et al. Eur. J. Org. Chem. 2305 (1998) and references cited therein). These groups can be introduced in the reaction of the amine with the activated acyl derivatives such as anhydrides, halogenides, active esters, etc. in the presence of a base, except for the dithiasuccinoyl group which can be formed via a two-step sequence (ethoxythiocarbonylation followed by the cyclization with chlorocarbonylsulfenyl chloride, see e.g. Meinjohanns et al. JCS Perkin Trans. 1 405 (1995)).

The N-protected isolactosamine derivatives obtained may be brought to reactions for protecting OH-groups. For instance, peracylation can be conducted with an acylating agent such as halogenides, anhydrides or active derivatives of carboxylic acids (e.g. imidazolide, thioester, silyl ester, vinyl ester, tetrazolide, ortoester, hydroxy-benztriazolyl ester, etc.) in the presence of a base like pyridine, triethylamine, diisopropyl ethylamine, dimethylaminopyridine, etc. in organic solvents such as DCM, chloroform, THF, dioxane, acetonitrile, etc. or mixture thereof at −20-80° C. These 1-O-acylglycosides may be then used either for direct glycosidation or for preparing further activated glycosyl donors such as halogenides or thioglycosides. Selective removal of the 1-O-acyl group (e.g. with water in the presence of Lewis or Bronsted acid, or by treatment with a primary amine such as ammonia, hydrazine, alkyl/benzyl amines) results in the protected glycosyl hemiacetal which may be converted in a trichloroacetimidate donor, for example. These donors are now ready to be coupled to the desired sugar moiety as acceptor.

The N-protected isolactosamine may be selectively protected among the hydroxyl groups. Thus a reaction with an aromatic aldehyde or dimethyl acetal thereof under acid catalysis may lead to the 4,6-O-acetal on the galactose portion. Bulky protecting groups like silyl (e.g. t-butyldimethylsilyl, t-butyldiphenylsilyl, triisopropylsilyl, etc.) or trityl may be applicable for masking the primary hydroxyls selectively. Various esters may be good donors for enzymes to transacylate yielding 6,6′-di-O-acyl derivatives.

Other features of the invention will become apparent in the course of the following descriptions of exemplary embodiments which are given for illustration of the invention and are not to be limiting thereof.

Experimental

### EXAMPLE 1

### a) Donors Used for Synthesis:

2,3,4,6-tetra-O-acetyl-α-D-galactopyranosyl trichloroacetimidate (Schmidt et al. Liebigs Ann. Chem. 1343 (1984));

2,3,4,6-tetra-O-benzoyl-α-D-galactopyranosyl trichloroacetimidate (Rio et al. Carbohydr. Res. 219, 71 (1991)).

### b) Acceptors Used for Synthesis:

benzyl 2-acetamido-2-deoxy-4,6-O-isopropylidene-β-D-glucopyranoside (Rana et al. Carbohydr. Res. 96, 231 (1981));

benzyl 2-benzyloxycarbonylamino-2-deoxy-4,6-O-isopropylidene-α-D-glucopyranoside (Imoto et al. Bull. Chem. Soc. Jpn. 60, 2205 (1987));

benzyl 2-trichloroacetamido-2-deoxy-4,6-O-benzylidene-α-D-glucopyranoside (prepared analogously as benzyl 2-trichloroacetamido-2-deoxy-4,6-O-benzylidene-β-D-glucopyranoside according to Blatter et al. Carbohydr. Res. 260, 189 (1994);

¹-H-NMR (CDCl₃, 300 MHz) δ 2.62 (bs, 1H, OH), 3.62 (dd, 1H, J=9.3 9.3 Hz, H-4), 3.79 (dd, 1H, J=10.2 10.2 Hz, H-6a), 3.91 (ddd, 1H, J=4.5 9.3 10.2 Hz, H-5), 4.06 (dd, 1H, J=9.3 9.3 Hz, H-3), 4.18 (ddd, 1H, J=3.6 8.7 9.3 Hz, H-2), 4.29 (dd, 1H, J=4.5 10.2 Hz, H-6b), 4.54 (d, 1H, J=11.7 Hz, CH₂Ph), 4.75 (d, 1H, J=11.7 Hz, CH₂Ph), 5.01 (d, 1H, J=3.6 Hz, H-1), 5.57 (s, 1H, PhCH), 6.95 (d, 1H, J=8.7 Hz, NH), 7.31-7.51 (m, 10H, Ph). ¹³C-NMR (CDCl₃, 75.45 MHz) δ 55.3 (C-2), 62.8 (C-5), 68.7 (C-6), 69.9 (C-3), 70.0 (CH₂Ph), 81.6 (C-4), 92.0 (CCl₃), 96.2 (C-1), 102.0 (PhCH), 126.2 (Ph), 128.4-129.4 (Ph), 136.2 (Ph), 136.8 (Ph), 162.2 (COCCl₃);

benzyl 2-trichloroacetamido-2-deoxy-4,6-O-isopropylidene-β-D-glucopyranoside (prepared analogously as benzyl 2-trichloroacetamido-2-deoxy-4,6-O-benzylidene-β-D-glucopyranoside according to Blatter et al. Carbohydr. Res. 260, 189 (1994);

¹H-NMR (CDCl₃, 300 MHz) δ 1.43 (s, 3H, CH₃), 1.52 (s, 3H, CH₃), 3.32 (ddd, 1H, J=5.6 9.9 10.5 Hz, H-5), 3.53-3.63 (m, 2H, H-2,4), 3.84 (dd, 1H, J=10.5 10.5 Hz, H-6a), 3.98 (dd, 1H, J=5.6 10.5 Hz, H-6b), 4.11 (dd, 1H, J=8.7 9.9 Hz, H-3), 4.60 (d, 1H, J=11.7 Hz, OCH₂Ph), 4.84 (d, 1H, J=8.4 Hz, H-1), 4.87 (d, 1H, J=11.7 Hz, OCH₂Ph), 6.84 (d, 1H, J=7.7 Hz, NH), 7.29-7.36 (m, 5H, Ph). ¹³C-NMR (MeOH-d₄, 300 MHz) δ 18.2, 28.3, 58.6, 61.9, 67.4, 70.8, 71.1, 74.5, 93.0, 99.7, 100.6, 127.6, 127.7, 128.2, 137.5, 163.5; m.p.: 209-210° C. (EtOAc/n-hexane);

1-O-benzoyl-2-benzyloxycarbonylamino-2-deoxy-4,6-O-isopropylidene-β-D-glucopyranose: to a suspension of 2-benzyloxycarbonylamino-2-deoxy-4,6-O-isopropylidene-β-D-glucopyranose (Hasegawa et al. Carbohydr. Res. 63, 91 (1978); 5.0 g) and 1-benzoyloxy-1H-benztriazole (3.89 g) in DCM (50 ml) at 5-10° C., was added Et₃N (5.88 ml) drop wise over 20 minutes. The mixture was stirred at RT overnight, then the reaction solution was diluted with 50 ml of DCM and extracted with sat. aq. bicarbonate solution. The organic phase was washed once with brine (100 ml), dried (MgSO₄), filtered and concentrated to give the title compound as a white solid (6.2 g, only β-anomer). ¹H-NMR (CDCl₃, 300 MHz) δ 1.45 (s, 3H, CH₃), 1.53 (s, 3H, CH₃), 2.81 (d, 1H, J=2.7 Hz, OH), 3.47 (ddd, 1H, J=6.0 9.9 9.9 Hz, H-5), 3.69 (dd, 1H, J=9.9 9.9 Hz, H-3), 3.80 (dd, 2H, J=9.9 9.9 Hz, H-6a and H-4), 3.95-4.04 (m, 2H, H-2,6b), 4.93 (d, 1H, J=9.0 Hz, NH), 5.02 (d, 1H, J=12.6 Hz, OCH₂Ph), 5.10 (d, 1H, J=12.6 Hz, OCH₂Ph), 5.85 (d, 1H, J=8.7 Hz, H-1), 7.16-8.08 (m, 10H, Ph). ¹³C-NMR (CDCl₃, 75.45 MHz) δ 19.0 (CH₃), 28.9 (CH₃), 54.4 (C-2), 61.7 (C-6), 67.9 (C-5 and OCH₂Ph), 72.1 (C-4), 73.7 (C-3), 93.7 (C-1), 99.9 (C(CH₃)₂), 127.8-128.6 (Ph), 130.2 (Ph), 133.8 (Ph), 135.9 (Ph), 156.6 (NHCOO), 165.1 (COOPh).

### Example 2

### a) Typical Glycosylation Procedure:

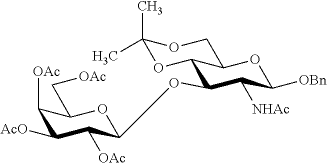

2,3,4,6-Tetra-O-acetyl-α-D-galactopyranosyl trichloroacetimidate (175 g) and benzyl 2-acetamido-2-deoxy-4,6-O-isopropylidene-β-D-glucopyranoside (100 g) were dissolved in dichloromethane (500 ml). To the resulting suspension BF₃-etherate (6 ml) was added and the reaction mixture was stirred at rt for 24 hours. Completion of the reaction was checked by TLC. The reaction mixture was extracted with sat. NaHCO₃ (150 ml), 0.5% NaHCO₃ (3×100 ml) and sat. NaCl (100 ml) solutions, the organic phase was dried and the solvent was evaporated giving a thick syrup, which was taken up in hot acetone for crystallization. 106 g of white crystals.

¹H-NMR (CDCl₃, 300 MHz) δ 1.42 (s, 3H, CH₃C), 1.53 (s, 3H, CH₃C), 1.89 (s, 3H, NHAc), 1.97 (s, 3H, OAc), 2.01 (s, 3H, OAc), 2.05 (s, 3H, OAc), 2.14 (s, 3H, OAc), 3.00 (m, 1H, H-2), 3.39 (m, 1H), 3.74 (dd, 1H, J=9.0 9.0 Hz), 3.83 (m, 2H), 3.95 (dd, 1H, J=5.4 10.8 Hz), 4.08 (m, 1H), 4.18 (dd, 1H, J=6.0 11.1 Hz), 4.52 (m, 1H), 4.53 (d, 1H, J=11.7 Hz, OCH₂Ph), 4.83 (d, 1H, J=11.7 Hz, OCH₂Ph), 4.87 (d, 1H, J=7.8 Hz, H-1), 4.95 (dd, 1H, J=3.3 10.5 Hz, H-3′), 5.13 (dd, 1H, J=8.1 10.5 Hz, H-2′), 5.23 (d, 1H, J=8.1 Hz, H-1′), 5.35 (dd, 1H, J≦1.0 and =3.3 Hz, H-4′), 5.64 (d, 1H, J=7.2 Hz, NH), 7.27-7.36 (m, 5H, Ph).

¹³C-NMR (CDCl₃, 75.45 MHz) δ 19.0 (CH₃), 20.5 (CH₃CO), 20.6 (2×CH₃CO), 20.8 (CH₃CO), 23.6 (NHCOCH₃), 29.1 (CH₃), 58.3 (C-2), 61.3 (C-6/6′), 62.0 (C-6/6′), 66.4 (C-4′), 66.9, 69.6, 70.6, 71.2 (C-3′), 71.7 (OCH₂Ph), 73.9, 76.8, 98.6 (C-1′), 99.3 (C(CH₃)₂), 99.8 (C-1), 128.0 (Ph), 128.1(Ph), 128.4 (Ph), 137.0 (Ph), 164.6 (NHCOCH₃), 169.6 (COCH₃), 170.1 (COCH₃), 170.2 (COCH₃), 170.4 (COCH₃).

M.p.: 194-198° C.

b)

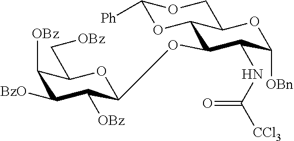

From 2,3,4,6-tetra-O-benzoyl-α-D-galactopyranosyl trichloroacetimidate and benzyl 2-trichloroacetamido-2-deoxy-4,6-O-benzylidene-α-D-glucopyranoside as described in Example 2a.

¹H-NMR (CDCl₃, 300 MHz) δ 3.80 (dd, 1H, J=9.9 9.9 Hz, H-6a), 3.89-4.02 (m, 3H, H-4,5,5′), 4.26-4.36 (m, 4H, H-2,3,6b,6a′), 4.47-4.53 (m, 2H, CH₂Ph, H-6′b), 4.70 (d, 1H, J=11.7 Hz, CH₂Ph), 4.98 (d, 1H, J=3.3 Hz, H-1), 5.13 (d, 1H, J=7.8 Hz, H-1′), 5.44 (dd, 1H, J=3.3 10.2 Hz, H-3′), 5.63 (s, 1H, PhCH), 5.69 (dd, 1H, J=7.8 10.2 Hz, H-2′), 5.89 (dd, 1H, J=0.9, 3.3 Hz, H-4′), 5.73 (d, 1H, J=8.4 Hz, NH), 7.16-8.05 (m, 30H, Ph).

¹³C-NMR (CDCl₃, 75.45 MHz) δ 54.6 (C-2), 61.3 (C-6′), 63.1 (C-5), 67.6 (C-4′), 68.8 (C-6), 70.1 (CH₂Ph), 70.4 (C-2′), 71.0 (C-5′), 71.9 (C-3′), 75.3 (C-3), 80.1 (C-4), 92.2 (CCl₃), 96.3(C-1), 100.1 (C-1′), 101.6 (PhCH), 125.9 (Ph), 127.9-129.9 (Ph), 133.1-133.4 (Ph), 136.1 (Ph), 136.7 (Ph), 161.5 (COCl₃), 165.1 (COPh), 165.4 (COPh), 165.6 (COPh), 165.8 (COPh).

c)

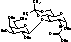

From 2,3,4,6-tetra-O-benzoyl-α-D-galactopyranosyl trichloroacetimidate and benzyl 2-benzyloxycarbonylamino-2-deoxy-4,6-O-isopropylidene-α-D-glucopyranoside as described in Example 2a.

¹H-NMR (CDCl₃, 300 MHz) δ 1.38 (s, 3H, CH₃C), 1.46 (s, 3H, CH₃C), 3.58-3.89 (m, 4H, H-4, 5, 6ab), 3.96 (dd, 1H, J=3.6 9.3 Hz, H-2), 4.17 (ddd, 1H, J=1.2 5.7 7.8 Hz, H-5′), 4.26 (d, 1H, J=12.0 Hz, OCH₂Ph), 4.28 (dd, 1H, J=7.8 11.1 Hz, H-6a′), 4.34 (dd, 1H, J=9.3 9.3 Hz, H-3), 4.54 (d, 1H, J=12.0 Hz, OCH₂Ph), 4.67 (dd, 1H, J=5.7 11.1 Hz, H-6b′), 4.73 (d, 1H, J=3.6 Hz, H-1), 4.75 (d, 1H, J=12.0 Hz, NHCOOCH₂Ph), 5.02 (d, 1H, J=7.8 Hz, H-1′), 5.05 (d, 1H, J=12.0 Hz, NHCOOCH₂Ph), 5.43 (dd, 1H, J=3.6 10.5 H-3′), 5.63 (dd, 1H, J=7.8 10.5 Hz, H-2′), 5.90 (dd, 1H, J=1.2 3.6 Hz, H-4′), 7.02-8.06 (m, 30H, Ph).

¹³C-NMR (CDCl₃, 75.45 MHz) δ 19.0 (CH₃), 29.1 (CH₃), 54.3 (C-2), 61.6 (C-6′), 62.3 (C-6), 63.6 (C-5), 66.6 (C(O)OCH₂Ph), 67.9 (C-4′), 69.6 (OCH₂Ph), 70.9 (C-2′), 71.4 (C-3), 71.9 (C-3′), 74.5 (C-5′), 78.0 (C-4), 97.5 (C-1), 99.7 (C(CH₃)₂), 101.6 (C-1′), 127.7-130.3 (Ph), 133.0-133.3 (Ph), 136.4-137.8 (Ph), 155.6 (NHCOO), 164.7 (COPh), 165.4 (COPh), 165.5 (COPh), 166.0 (COPh).

d)

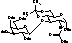

From 2,3,4,6-tetra-O-acetyl-α-D-galactopyranosyl trichloroacetimidate and benzyl 2-benzyloxycarbonylamino-2-deoxy-4,6-O-isopropylidene-α-D-glucopyranoside as described in Example 2a.

¹H-NMR (CDCl₃, 300 MHz) δ 1.41 (s, 3H, CH₃C), 1.52 (s, 3H, CH₃C), 1.93 (s, 3H, CH₃CO), 1.96 (s, 3H, CH₃CO), 2.02 (s, 3H, CH₃CO), 2.13 (s, 3H, CH₃CO), 3.66-3.83 (m, 5H, H-4,5,5′6ab), 3.99 (m, 1H, H-2), 4.09 (dd, 1H, J=4.5 11.1 Hz, H-6a′), 4.17 (dd, 1H, J=5.7 11.1 Hz, H-6b′), 4.44 (d, 1H, J=11.7 Hz, OCH₂Ph), 4.64 (d, 1H, J=8.1 Hz, H-1′), 4.66 (d, 1H, J=11.7 Hz, OCH₂Ph), 4.85-4.90 (m, 2H, H-1,3), 4.93 (dd, 1H, J=3.3 10.5 Hz, H-3′), 5.0 (d, 1H, J=11.7 Hz, NHCOOCH₂Ph), 5.17 (d, 1H, J=11.7 Hz, NHCOOCH₂Ph), 5.18 (dd, 1H, J=8.1 10.5 Hz, H-2′), 5.33 (dd, 1H, J=0.9 3.3 Hz, H-4′), 7.17-7.36 (m, 10H, Ph).

¹³C-NMR (CDCl₃, 75.45 MHz) δ 19.0 (CCH₃), 20.5-20.6 (4×CH₃CO), 29.0 (CCH₃), 53.9 (C-2), 61.1 (C-6′), 62.2 (C-6), 63.4 (C-5), 66.9 (C-4′), 67.1 (C(O)OCH₂Ph), 69.4 (C-2′), 69.9 (OCH₂Ph), 70.5 (C-3), 71.1 (C-3′), 73.8 (C-5′), 78.6 (C-4), 97.4 (C-1), 99.5 (C(CH₃)₂), 101.2 (C-1′), 128.1-129.0 (Ph), 136.2-136.6 (Ph), 155.5 (NHCOO), 169.2 (COMe), 170.2 (COMe), 170.3 (2×COMe).

e)

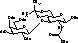

From 2,3,4,6-tetra-O-acetyl-α-D-galactopyranosyl trichloroacetimidate and 1-O-benzoyl-2-benzyloxycarbonylamino-2-deoxy-4,6-O-isopropylidene-β-D-glucopyranose as described in Example 2a.

¹H-NMR (CDCl₃, 300 MHz) δ 1.43 (s, 3H), 1.53 (s, 3H), 1.93 (s, 3H), 1.96 (s, 3H), 2.03 (s, 3H), 2.15 (s, 3H), 3.44-3.52 (m, 1H), 3.74-3.99 (m, 6H), 4.09-4.22 (m, 2H), 4.70 (d, 1H, J=7.8 Hz, H-1′), 4.97 (dd, 1H, J=3.6 10.5 Hz, H-3′), 5.03-5.11 (m, 3H, NH+OCH₂Ph), 5.20 (dd, 1H, J=7.8 10.5 Hz H-2′), 5.36 (d, 1H, J=3.6 Hz, H-4′), 6.01 (d, 1H, J=8.1 Hz, H-1), 7.15-8.04 (m, 10H, Ph). ¹³C-NMR (CDCl₃, 75.45 MHz) δ 18.9, 20.5-20.6 (4C), 29.0, 56.0, 61.0, 61.7, 66.9 (4C), 67.6, 69.3, 70.5, 71.0, 73.0, 93.1, 99.6, 127.9-130.1, 133.8, 135.9, 155.5, 164.9, 169.4, 170.1, 170.2, 170.3.

f)

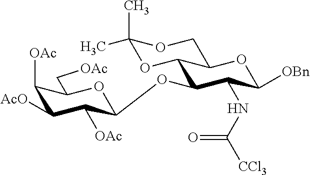

From 2,3,4,6-tetra-O-acetyl-α-D-galactopyranosyl trichloroacetimidate and benzyl 2-trichloroacetamido-2-deoxy-4,6-O-isopropylidene-β-D-glucopyranoside as described in Example 2a.

¹H-NMR (CDCl₃, 300 MHz, main rotamer at 25° C. listed) δ 1.42 (s, 3H, CH₃), 1.54 (s, 3H, CH₃), 1.90 (s, 3H, CH₃), 1.96 (s, 3H, CH₃), 2.04 (s, 3H, CH₃), 2.14 (s, 3H, CH₃), 3.31-3.46 (m, 2H), 3.77-3.87 (m, 3H), 3.95-4.14 (m, 3H), 4.41 (dd, 1H, J=9.3 9.3 Hz), 4.55 (d, 1H, J=11.1 Hz, OCH₂Ph), 4.79 (d, 1H, J=8.1 Hz, H-1), 4.84 (d, 1H, J=11.1 Hz, OCH₂Ph), 4.96 (dd, 1H, J=3.9 9.9 Hz, H-3′), 5.14-5.20 (m, 2H, H-1′, H-2′), 5.34 (d, 1H, J=3.9 Hz, H-4′), 7.03 (d, 1H, J=7.2 Hz, NH), 7.29-7.30 (m, 5H, Ph).

¹³C-NMR (CDCl₃:MeOH-d₄ (1:1), 75.45 MHz) δ 19.8 (3C), 20.1, 23.1, 30.1, 56.3, 60.6, 61.3, 66.7, 67.9, 68.4, 70.2, 70.3, 70.5, 75.5, 81.1, 92.4, 98.8, 99.0, 100.2, 127.4, 127.6, 127.8, 136.4, 162.2, 170.0, 170.1, 170.2, 170.5.

M.p. 182-184° C. (EtOAc/n-hexane)

### Example 3

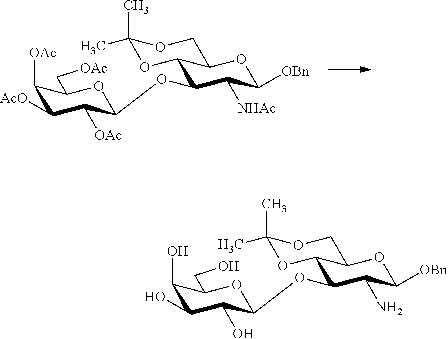

To a solution of NaOH (30 g) in water (120 ml) the protected disaccharide was added in portions. The suspension was heated to reflux and stirred for 40 hours. After cooling THF (240 ml) was added, the phases were separated, the organic phases was dried and concentrated giving a solidified foam.

¹H-NMR (MeOH-d₄, 300 MHz) δ 1.40 (s, 3H), 1.53 (s, 3H), 2.85 (dd, 1H, J=8.4 9.3 Hz, H-2), 3.47 (dd, 1H, J=3.3 9.6 Hz), 3.52 (ddd, 1H, J=1.2 6.3 6.3 Hz), 3.61 (dd, 1H, J=8.1 9.6 Hz), 3.66 (dd, 1H, J=9.3 9.3 Hz), 3.72-3.87 (m, 6H), 3.92 (dd, 1H, J=5.4 10.4 Hz), 4.43 (d, 1H, J=8.4 Hz, H-1), 4.62 (d, 1H, J=11.7 Hz, OCH₂Ph), 4.85 (d, 1H, J=11.7 Hz, OCH₂Ph), 7.25-7.40 (m, 5H) (H1′-signal hidden under solvent signal).

¹³C-NMR (MeOH-d₄, 75.45 MHz) δ 19.4, 29.4, 58.5, 62.2, 63.2, 68.8, 70.2, 72.0, 72.3, 74.0, 74.9, 77.1, 81.6, 101.2, 104.4, 104.6, 129.0, 129.3, 129.5, 138.8.

### Example 4

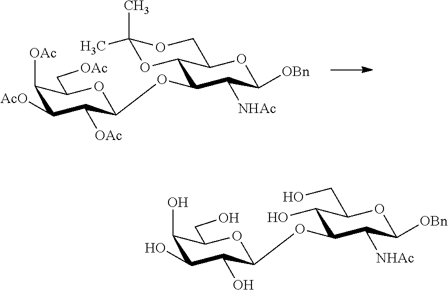

Solid NaOMe (150 mg) was added to a suspension of the protected disaccharide (50 g) in 250 mL of MeOH at RT. After 2.5 hours pTsOH monohydrate (0.85 g) and water (5 mL) were added. After 15 minutes a gel-like solid started to form. Another portion of MeOH (150 mL) was added and the slurry was heated to 40° C. for 3 hours. The slurry was allowed to cool to RT then the suspension was stirred in the cold room over night, filtered. The solid was dried to give 30.9 g of product.

¹H-NMR (D₂O, 300 MHz) δ 1.77 (s, 3H), 3.26-3.79 (m, 12H), 4.19 (d, 1H, J=7.5 Hz), 4.39 (d, 1H, J=8.1 Hz), 4.49 (d, 1H, J=12.3 Hz), 4.71 (d, 1H, J=12.3 Hz), 7.17-7.30 (m, 5H).

¹³C-NMR (D₂O, 75.45 MHz) δ 22.3, 54.7, 60.9, 61.1, 68.6, 68.8, 70.8, 71.6, 72.6, 75.4, 75.5, 82.5, 99.8, 103.6, 128.6, 128.7, 128.9, 135.8, 174.6.

M.p.: 203-205° C. (MeOH/water)

### Example 5

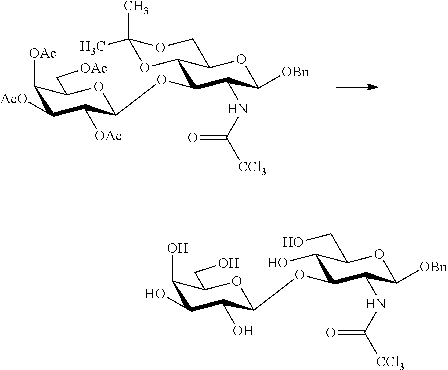

The procedure described in Example 3 was followed.

¹H-NMR (MeOH-d₄, 300 MHz, selected signals, one benzylic proton under solvent signal) δ 4.35 (d, 1H, J=7.8 Hz), 4.64 (d, 1H, J=12.0 Hz), 4.71 (d, 1H, J=8.1 Hz), 7.25-7.36 (m, 5H).

¹³C-NMR (MeOH-d₄, 75.45 MHz) δ 57.1, 61.4, 61.5, 69.0, 69.4, 70.6, 71.2, 73.4, 75.9, 76.4, 81.7, 92.9, 99.5, 103.7, 127.6, 127.9, 128.2, 137.5, 163.2.

M.p. 184-185° C.

### Example 6

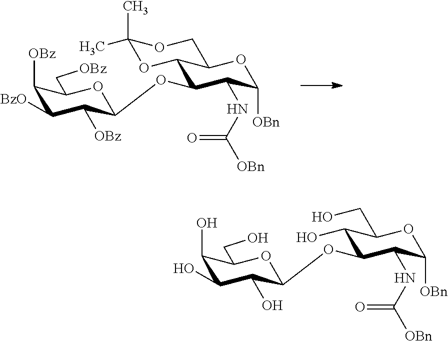

The protected disaccharide (13.1 g) was dissolved in DCM/MeOH (2.5:1, 140 ml) and 350 mg of NaOMe was added. Stirred at RT for 18 h when 60 mL of MeOH was added and the solution neutralized with IR-Amberlite 120. The resins were filtered off and the filtrate concentrated. The obtained syrup was dissolved in DCM/MeOH (1:1, 100 mL) and 10 mL of 30% aq. HClO₄ was added. After totally 30 min., the formed solid was filtered off and dried. Re-crystallization from hot EtOH gave 3.36 g of the title compound.

¹H-NMR (MeOH-d4, 300 MHz) δ 3.39-3.53 (m, 4H), 3.58-3.85 (m, 8H), 4.36 (d, 1H, J=7.5 Hz, H-1′), 4.47 (d, 1H, J=11.7 Hz, OCH₂Ph), 4.69 (d, 1H, J=11.7 Hz, OCH₂Ph), 4.84 (d, 1H, H-1, under solvent peak), 4.95 (d, 1H, J=12.6 Hz, NHCOOCH₂Ph), 5.07 (d, 1H, J=12.6 Hz, NHCOOCH₂Ph), 7.23-7.35 (m, 10H, Ph).

¹³C-NMR (DMSO-d₆, 75.45 MHz) δ 54.2, 60.4, 60.5, 65.3, 68.0, 68.1, 68.7, 70.6, 72.8, 73.3, 75.6, 80.4, 96.0, 103.1, 127.4-128.4, 137.0, 137.6, 156.2.

M.p.: 220-223° C.

### Example 7

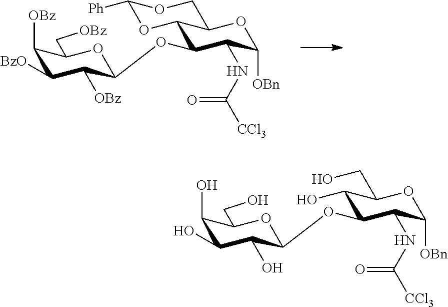

The procedure described in Example 3 was followed.

¹H-NMR (MeOH-d₄, 300 MHz) δ 3.44 (dd, 1H, J=3.3 9.9 Hz), 3.51-3.57 (m, 3H), 3.65-3.91 (m, 6H), 4.04 (dd, 1H, J=3.6 10.5 Hz), 4.14 (dd, 1H, J=8.4 10.5 Hz), 4.46 (d, 1H, J=7.5 Hz, H-1′), 4.53 (d, 1H, J=11.7 Hz, CH₂Ph), 4.76 (d, 1H, J=11.7 Hz, CH₂Ph), 4.99 (d, 1H, J=3.6 Hz, H-1), 7.27-7.41 (m, 5H, Ph).

¹³C-NMR (MeOH-d₄, 75.45 MHz) δ 56.4, 62.5 (2C), 70.2, 70.3, 70.7, 72.5, 73.7, 74.7, 77.1, 80.6, 93.7, 96.6, 104.6, 129.0, 129.4, 129.6, 138.4, 162.4.

### Example 8

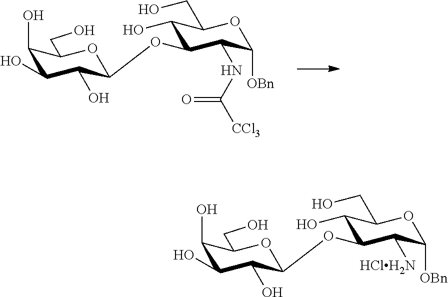

To a solution of the N-trichloroacetamido derivative (1.0 g) in pre-heated MeOH (5 ml, 45° C.) was added 2M aq. NaOH solution (1.5 equiv.). The solution was stirred for 4 hours and then cooled to RT followed by adding 1M aq. HCl to adjust the pH to 4. The solution was evaporated to dryness and re-dissolved in MeOH (2.5 ml) and the mixture was cooled at 0-5° C. for 1 hour before filtrated. To the filtrate was added isopropanol (3 ml) to precipitate out the product as an amorphous solid (0.65 g, 80%).

¹H-NMR (D₂O, 300 MHz) δ 3.55-3.77 (m, 10H), 3.92 (d, 1H, J=3.0 Hz, H-4), 4.11 (dd, 1H, J=8.1 10.2 Hz), 4.56 (d, 1H, J=7.5 Hz, H-1′), 4.65 (d, 1H, J=11.4 Hz, OCH₂Ph), 4.77 (d, 1H, J=11.4 Hz, OCH₂Ph), 5.27 (d, 1H, J=3.6 Hz, H-1), 7.41-7.47 (m, 5H, Ph).

¹³C-NMR (D₂O, 75.45 MHz) δ 53.1, 60.1, 61.0, 68.0, 68.5, 70.2, 70.9, 72.0, 72.5, 75.9, 80.0, 94.6, 103.0, 128.8, 128.9, 129.0, 136.6.

### Example 9

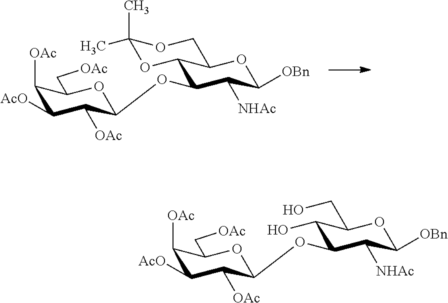

To a solution of the protected disaccharide (5 g) in DCM (20 ml) at 0-5° C. was added diluted aq. HClO₄ (1 part 70% aq. HClO₄ mixed with 1 part water; 0.75 ml). The mixture was stirred vigorously for 1 hour when sat. aq. sodium bicarbonate solution was added slowly (20 ml). The mixture was allowed to reach RT during 1 hour with stirring and then poured in a funnel. The layers were separated and the organic extract was dried over sodium sulphate, filtered and evaporated to dryness to get a white solid (4.33 g, 92%). ¹H-NMIR (CDCl₃, 300 MHz) δ 1.93 (s, 3H, CH₃C), 1.97 (s, 3H, CH₃C), 2.05 (s, 3H, CH₃C), 2.06 (s, 3H, CH₃C), 2.14 (s, 3H, CH₃C), 3.05-3.13 (m, 1H), 3.38-3.54 (m, 2H), 3.78 (dd, 1H, J=5.1 12.0 Hz), 3.92 (dd, 1H, J=3.6 12.0 Hz), 3.99 (m, 1H), 4.09-4.12 (m, 2H), 4.40 (dd, 1H, J=7.8 10.2 Hz), 4.53 (d, 1H, J=8.1 Hz), 4.58 (d, 1H, J =12.0 Hz), 4.85 (d, 1H, J=12.0 Hz), 4.96-5.03 (m, 2H), 5.20 (dd, 1H, J=7.8 10.2 Hz), 5.35 (d, 1H, J=3.3 Hz), 5.83 (d, 1H, J=6.9 Hz), 7.27-7.38 (m, 5H).

¹³C-NMR (CDCl₃, 75.45 MHz) δ 20.5, 20.5, 20.6, 20.7, 23.6, 57.2, 61.5, 62.6, 66.9, 68.7, 70.0, 70.7, 71.0, 71.6, 75.3, 83.3, 98.9, 101.3, 128.0 (2C), 128.2, 128.8, 137.1, 169.2, 170.0, 170.1, 170.5, 170.8.

### Example 10

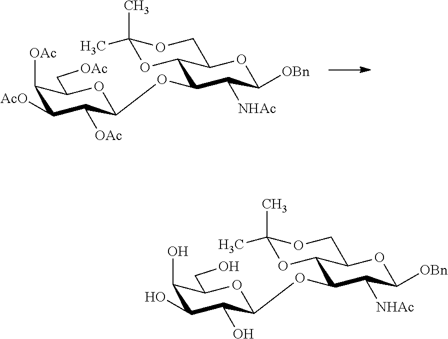

To a suspension of fully protected disaccharide derivative (10 g) in MeOH (50 ml) was added solid NaOMe (40 mg). The mixture was stirred over night at RT. When TLC indicated complete reaction, IR-Amberlite 120H was added to adjust pH to 5-6, the resin was filtrated off and washed with some MeOH. The filtrate was evaporated to dryness and dried under reduced pressure to give the deacetylated product as a solid (7.15 g, 95 %).

¹H-NMR (MeOH-d₄, 300 MHz, selected signals) δ 1.40 (s, 3H), 1.55 (s, 3H), 1.94 (s, 3H), 4.38 (d, 1H, J=7.8 Hz), 4.51 (d, 1H, J=8.1 Hz), 4.57 (d, 1H, J=12.3 Hz), 4.81 (d, 1H, J=12.3 Hz), 7.25-7.34 (m, 5H).

¹³C-NMR (MeOH-d₄, 75.45 MHz) δ 19.4, 23.3, 29.4, 56.6, 62.3, 63.1, 68.4, 70.1, 71.4, 71.9, 73.9, 74.7, 77.0, 77.3, 101.2, 102.5, 103.6, 128.8, 128.9, 129.4, 138.9, 173.8.

M.p.: 235-236° C. (diisopropyl ether/MeOH)

### Example 11

a)

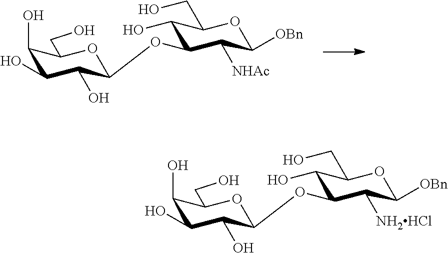

The mixture of disaccharide (5 g) and 4M aq. KOH solution (16 ml) was heated to reflux for 24 hrs. The thick solution was allowed to cool down to RT and 4 ml of water was added. The solution was neutralized with 2M aq. HCl solution giving a precipitation which was filtered off. The filtrate was concentrated and re-dissolved in MeOH (50 ml) and heated to 50° C. Some additional insoluble material was filtered off and 25 ml of isopropanol was added. The solution was allowed to cool down with stirring in the cold room overnight. The formed solid was filtered off and dried in an oven at reduced pressure at 40° C. to give 2.75 g of the disaccharide as the hydrochloride.

b)

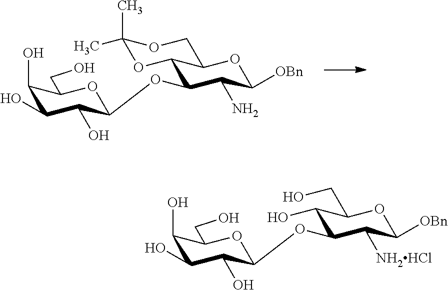

Disaccharide (8.2 g) was dissolved in a mixture of 2M HCl solution (11.9 ml) and water (30.5 ml), and solution was stirred at rt for 16 hours. The solvent was removed under vacuum to give a thick syrup (quant.)

¹H-NMR (D₂O, 300 MHz) δ 2.65 (m, 1H, H-2), 3.27-3.78 (m, 12H), 4.31 (d, 1H, J=8.4 Hz), 4.33 (d, 1H, J=7.3 Hz), 4.55 (d, 1H, J=11.3 Hz), 4.77 (d, 1H, J=11.3 Hz), 7.25-7.33 (m, 5H).

¹³C-NMR (D₂O, 75.45 MHz) δ 56.0, 60.9, 61.1, 71.1, 71.7, 72.7, 75.6, 75.8, 86.9, 101.7, 104.2, 128.7, 128.9, 129.2, 136.7.

M.p.: 166-168° C. (MeOH/EtOH)

### Example 12

Isolactosamine hydrochloride

Typical procedure for catalytic hydrogenolysis:

To compound of Example 11 (8.14 g) in 30 m of water was added 0.75 g of Pd/C (10%). The suspension stirred under H₂ for 6 hours. The catalyst was filtered off on a pad of Celite and the filtrate was concentrated under vacuum. The obtained thick syrup was taken up in methanol (50 ml) and the product was crystallized by addition of isopropanol (100 ml) giving rise to white crystals (5.54 g).

M.p.: 189-191° C. (dec.).

¹H and ¹³C resonance assignments in solution (600 MHz, D₂O) are listed in Table 1 below.

TABLE 1

| | | | multi- | | | |
|---|---|---|---|---|---|---|
| Ring | proton | δ (ppm) | plicity | J (Hz) | carbon | δ (ppm) |
| Gl (α) | H-1 | 5.21 | d | 3.6 | C-1 | 95.3 |
| | H-2 | 2.87 | dd | 10.0, 3.6 | C-2 | 57.1 |
| | H-3 | 3.72 | dd | 10.0, 8.7 | C-3 | 87.9 |
| | H-4 | 3.52 | dd | 10.0, 8.7 | C-4 | 71.0 |
| | H-5 | 3.86 | ddd | 10.0, 4.6, 2.5 | C-5 | 74.1 |
| | H-6x | 3.82 | dd | 12.5, 2.5 | C-6 | 63.3 |
| | H-6y | 3.79 | dd | 12.5, 4.6 | | |
| Gl (β) | H-1 | 4.61 | d | 8.3 | C-1 | 99.0 |
| | H-2 | 2.76 | dd | 9.4, 8.3 | C-2 | 59.5 |
| | H-3 | 3.57 | dd | 9.4, 8.7 | C-3 | 89.5 |
| | H-4 | 3.53 | dd | 9.3, 8.7 | C-4 | 71.0 |
| | H-5 | 3.48 | ddd | 9.3, 5.5, 1.7 | C-5 | 78.2 |
| | H-6x | 3.89 | dd | 12.6, 1.7 | C-6 | 63.4 |
| | H-6y | 3.75 | dd | 12.6, 5.5 | | |
| Gal | H-1 | 4.56 | d | 8.0 | C-1 | 106.7 |
| | | (4.53)* | | | | |
| | H-2 | 3.60 | dd | 9.8, 8.0 | C-2 | 73.8 |
| | H-3 | 3.68 | dd | 9.8, 3.2 | C-3 | 75.3 |
| | H-4 | 3.93 | d | 3.2 | C-4 | 71.2 |
| | H-5 | 3.74 | m | | C-5 | 78.1 |
| | H-6x | 3.76 | m | | C-6 | 63.6 |
| | H-6y | | | | | |

*corresponds to Gal connected to the β-glucose; other Gal protons are unresolved for Glu-α/Glu-β

### Example 13

Solid-state ¹³C-CP/MAS spectra were recorded at 600 MHz instrument by using a Varian Chemagnetics 3.2 mm NB HXY probe in ¹³C{¹H} double resonance mode. Solid state ¹³C chemical shifts are given relative to adamantane (d 38.55, 29.50 ppm). Contact time was 1 ms, repetition delay 60 s, MAS rate 10 kHz. SPINAL high power proton decoupling was performed. Solid state ¹³C-NMR spectrum is shown in FIG. 1.

1-10. (canceled)

11. Isolactosamine of formula 1 and salts thereof in the form of either an anomer or mixture anomers, hydrates or solvates of the free base or salts thereof

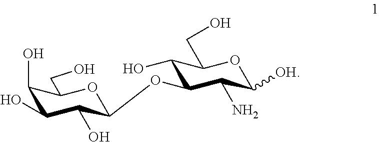

12. The isolactosamine and salts thereof according to claim 11 in crystalline form.

13. The isolactosamine salt according to claim 11, which is a hydrochloride salt.

14. A method for the preparation of isolactosamine or salts thereof according to claim 1, characterized in that a compound of general formula 2 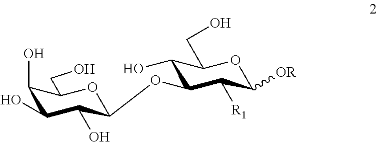
wherein R is selected from H and a group that can be removed by hydrogenolysis, and R₁ is selected from azido and —NHR₂ wherein R₂ is selected from H, optionally substituted benzyloxycarbonyl and optionally substituted benzyloxymethyl, provided if R is H then R₁ differs from —NH₂, or salts thereof in the form of either anomer or mixture thereof, hydrates or solvates of the free base and salts thereof, is subjected to catalytic hydrogenation/hydrogenolysis.

15. The method according to claim 14, wherein the catalytic hydrogenation/hydrogenolysis is performed in water or in aqueous alcohol in the presence of a catalyst comprising palladium, Raney nickel or other metal catalysts.

16. The method according to claim 15, wherein the catalytic hydrogenation/hydrogenolysis is performed in water.

17. The method according to claim 15, wherein the catalyst is palladium on charcoal or palladium black.

18. A compound of general formula 2 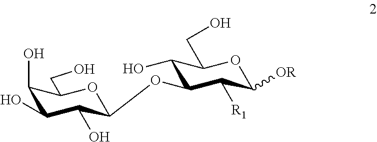
wherein R is selected from H and a group that can be removed by hydrogenolysis, and R₁ is selected from azido and —NHR₂ wherein R₂ is selected from H, optionally substituted benzyloxycarbonyl and optionally substituted benzyloxymethyl, or salts thereof in the form of either an anomer or mixture anomers, hydrates or solvates of the free base and salts thereof, provided if R is H then R₁ differs from —NH₂, and provided that benzyl 3-O-(β-D-galactopyranosyl)-2-deoxy-2-amino-α-D-glucopyranoside is excluded.

19. The compound according to claim 18 characterized by general formula 2′ 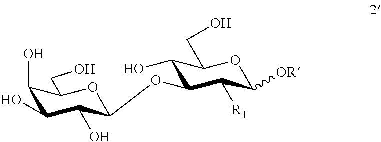
wherein R′ is a group that can be removed by hydrogenolysis, and R₁ is selected from azido and —NHR₂ wherein R₂ is selected from H, optionally substituted benzyloxycarbonyl and optionally substituted benzyloxymethyl.

20. The compound according to claim 19, wherein R′ is naphthylmethyl or benzyl optionally substituted by alkyl, halogen or phenyl, and R₁ is amino or benzyloxycarbonylamino.

21. The compound according to claim 20, wherein R′ is benzyl, 4-chlorobenzyl, 3-phenylbenzyl or 4-methylbenzyl.

22. The compound according to claim 21, wherein R′ is benzyl.

23. The compound according to claim 20, wherein R₁ is amino.

24. A method of using isolactosamine and salts thereof according to claim 11 for the preparation of lacto-N-biose containing oligosaccharides.

25. A method of synthesis of lacto-N-biose containing oligosaccharides, comprising the steps of:
a) masking of the amino group of isolactosamine according to claim 11 with a suitable protecting group,
b) protection of OH-groups,
c) activation of the anomeric position to obtain a lacto-N-biosyl donor, and
d) coupling the lacto-N-biosyl donor to a desired sugar moiety.
